(19) United States
(12) Patent Application Publication
(10) Pub. No.: US 2007/0021744 A1
(43) Pub. Date: Jan. 25, 2007

(54) Apparatus and method for performing ablation with imaging feedback

(76) Inventor: Francis M. Creighton IV, St. Louis, MO (US)

Correspondence Address: Bryan K. Wheelock, Suite 400, 7700 Bonhomme, St. Louis, MO, 63105, US

(21) Appl. No.: 11/483,396

(22) Filed: Jul. 7, 2006

Related U.S. Application Data
(60) Provisional application No. 60/697,322, filed on Jul. 7, 2005.

(51) Int. Cl. A61B 18/14

(52) U.S. Cl. 606/32; 606/41

(57) ABSTRACT

A method for ablating tissue is provided that comprises navigating an electrophysiology catheter to the site of the ablation, applying an ablating electrode on the electrophysiology catheter to the tissue to be ablated, applying energy to the electrode to ablate the tissue adjacent the electrode, ultrasonically imaging the site of the ablation, and re-ablating the tissue if the ultrasound imaging does not show a satisfactory ablation. Another embodiment of a method for ablation is provided that comprises forming a line of ablation by applying energy to an electrode to ablate the tissue adjacent the electrode, and ultrasonically imaging the line of ablation to locate points on the line that do not show satisfactory ablation. For points of unsatisfactory ablation, the method provides for navigating an electrophysiology catheter to at least one point on the line that does not show satisfactory ablation, and ablating the tissue at the point.

### CROSS-REFERENCE TO RELATED APPLICATIONS

This application claims the benefit of U.S. Provisional Patent Application Ser. No. 60/697,322, filed Jul. 7, 2005, the entire disclosure of which is incorporated herein by reference.

### FIELD

The present invention relates to the application of ablation techniques in forming lesions for treating arrhythmia or other conditions.

### BACKGROUND

Physicians typically require significant practice to develop the experience and skill required for performing accurate lesions by ablation, due to the difficulty in controlling and guiding the ablation catheter or device. Magnetically guided ablation catheters can improve ablation over that due to manually controlled catheter guidance by providing better control of the ablation catheter tip. Nevertheless, limited knowledge of the actual lesion formed on the surface of a beating heart can lead to significant uncertainty in ablation quality.

### SUMMARY

In accordance with a one aspect, embodiments of the invention provide a method for ablating tissue is provided that comprises navigating an electrophysiology catheter to the site of the ablation, applying an ablating electrode on the electrophysiology catheter to the tissue to be ablated, applying energy to the electrode to ablate the tissue adjacent the electrode, ultrasonically imaging the site of the ablation, and re-ablating the tissue if the ultrasound imaging does not show a satisfactory ablation. In another embodiment, a method of ablating tissue to form a line of ablation is provided that comprises forming a line of ablation by successively navigating an electrophysiology catheter to the site of the ablation, applying an ablating electrode on the electrophysiology catheter to the tissue to be ablated, applying energy to the electrode to ablate the tissue adjacent the electrode, and ultrasonically imaging the line of ablation to locate points on the line that do not show satisfactory ablation. Where points of unsatisfactory ablation are present, the method provides for navigating an electrophysiology catheter to at least one point on the line that does not show satisfactory ablation; applying an ablating electrode on the electrophysiology catheter to the tissue at the point; and applying energy to the electrode to ablate the tissue at the point. The step of navigating an electrophysiology catheter may comprise automatically identifying at least one point on the line of ablation that does not show satisfactory ablation through image processing; and automatically navigating the electrophysiology catheter to the identified point with a remote navigation system. The navigation of the electrophysiology catheter may be performed by a magnetic navigation system or by a mechanical navigation system.

In some embodiments, the ultrasonically imaging the line of ablation is performed with a separate device from the electrophysiology catheter. In one embodiment, the method of ablation and ultrasonically imaging the line of ablation is performed with the electrophysiology catheter. In either application, a method of ablating tissue is provided that calls for applying an ablating electrode on the electrophysiology catheter to the tissue to be ablated and applying energy to the electrode to ablate the tissue adjacent the electrode until ultrasonic imaging of the site indicates satisfactory ablation. Obtaining satisfactory ablation could entail alternately applying energy to the electrode to ablate the tissue adjacent the electrode and ultrasonically imaging the site until the ultrasonic imaging indicates satisfactory ablation.

In one embodiment, a method of treating arrhythmias is provided that comprises forming at least line of ablations to block errant signals causing the arrhythmias by ablating tissue until ultrasonic imaging indicates satisfactory ablation, and continuing the formation of lines until ultrasonic measurement of flow velocities in the heart indicates that the arrhythmias. The formation of successful ablation lines will establish flow velocities in the heart that indicate that the arrhythmia has been attenuated.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a side elevation view of one embodiment of a electrophysiology catheter in accordance with the principles of the present invention; and

FIG. 2 is a side elevation view of another embodiment comprising an ablation catheter and an ultrasound imaging catheter in accordance with the principles of the present invention.

### DETAILED DESCRIPTION

The following descriptions of the various embodiments are merely exemplary in nature and are in no way intended to limit the invention, its application, or uses.

The present invention provides for measurement of the ablation site during the ablation process, and to use that, possibly automatically, to control the duration of ablation. The present invention uses ultrasound imaging in combination with magnetic tip guidance to control ablation by imaging feedback, which is then used to redirect the tip of the ablation catheter and/or control the ablation duration. Intravascular ultrasound catheters have been developed for diagnostic and catheter guidance applications. However, because of ultrasound's sensitivity to tissue penetration as a function of frequency, multiple frequency ultrasound has an intrinsic ability to assess the hardness of ablated tissue. Multiple frequency ultrasound imaging allows for selecting specific frequencies for optimum tissue contrast resolution. The present invention uses ultrasound imaging in combination with magnetic tip guidance to image the ablation site and control ablation by imaging feedback, which can then be used during the ablation process to ensure a proper lesion is formed during the ablation procedure.

In one embodiment, a method for ablating tissue is provided that comprises navigating an electrophysiology catheter to the site of the ablation, applying an ablating electrode on the electrophysiology catheter to the tissue to be ablated, applying energy to the electrode to ablate the tissue adjacent the electrode, ultrasonically imaging the site of the ablation, and re-ablating the tissue if the ultrasound imaging does not show a satisfactory ablation. In another embodiment, a method of ablating tissue to form a line of ablation is provided that comprises forming a line of ablation by successively navigating an electrophysiology catheter to the site of the ablation, applying an ablating electrode on the electrophysiology catheter to the tissue to be ablated, applying energy to the electrode to ablate the tissue adjacent the electrode, and ultrasonically imaging the line of ablation to locate points on the line that do not show satisfactory ablation. Where points of unsatisfactory ablation are present, the method provides for navigating an electrophysiology catheter to at least one point on the line that does not show satisfactory ablation; applying an ablating electrode on the electrophysiology catheter to the tissue at the point; and applying energy to the electrode to ablate the tissue at the point. The step of navigating an electrophysiology catheter may comprise automatically identifying at least one point on the line of ablation that does not show satisfactory ablation through image processing; and automatically navigating the electrophysiology catheter to the identified point with a remote navigation system. The navigation of the electrophysiology catheter may be performed by a magnetic navigation system or by a mechanical navigation system.

In some embodiments, the ultrasonically imaging the line of ablation is performed with a separate device from the electrophysiology catheter. In one embodiment, the method of ablation and ultrasonically imaging the line of ablation is performed with the electrophysiology catheter. In either application, a method of ablating tissue is provided that calls for applying an ablating electrode on the electrophysiology catheter to the tissue to be ablated and applying energy to the electrode to ablate the tissue adjacent the electrode until ultrasonic imaging of the site indicates satisfactory ablation. Obtaining satisfactory ablation could entail alternately applying energy to the electrode to ablate the tissue adjacent the electrode and ultrasonically imaging the site until the ultrasonic imaging indicates satisfactory ablation.

Arrhythmias may be treated by such a method that comprises ablating tissue to block errant electrical signals causing the arrhythmias, until ultrasonic measurement of flow velocities in the heart indicates that the arrhythmias has been attenuated. In one method of treating arrhythmias, the method may comprise forming at least line of ablations to block errant signals causing the arrhythmias by ablating tissue until ultrasonic imaging indicates satisfactory ablation, and continuing the formation of lines until ultrasonic measurement of flow velocities in the heart indicates that the arrhythmias. The formation of successful ablation lines will establish flow velocities in the heart that indicate that the arrhythmia has been attenuated.

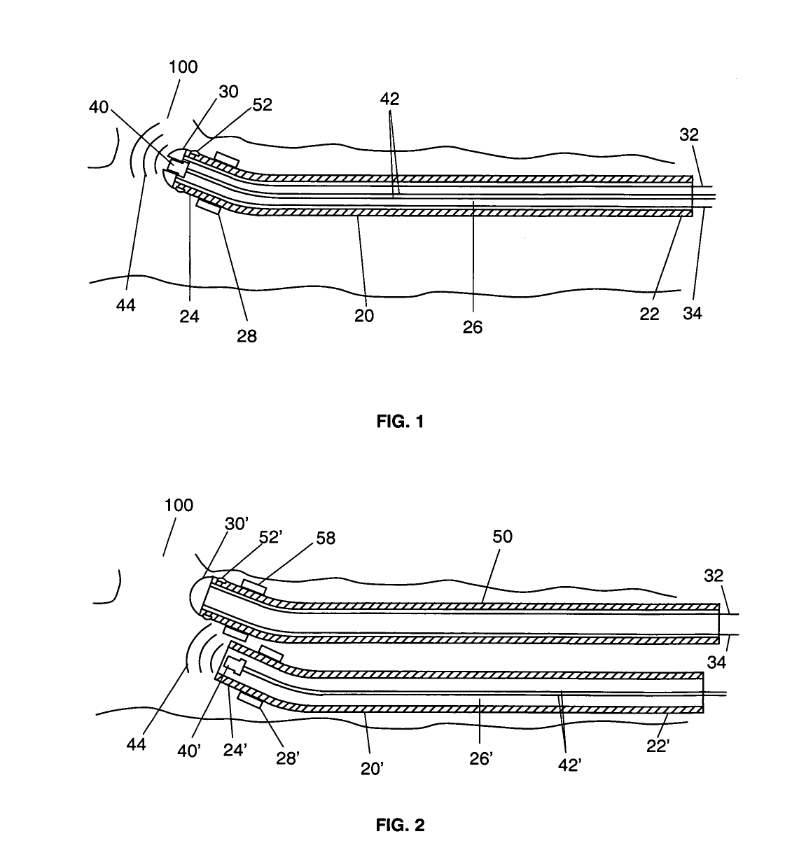

In one embodiment shown in FIG. 1, an electrophysiology catheter 20 comprises a proximal end 22 and a distal end 24 and a lumen 26 there between. The distal end 24 of the electrophysiology catheter 20 may further comprise a magnetically responsive element 28, where magnetic navigation systems are employed. The electrophysiology catheter 20 preferably comprises a tip 30 capable of operating as an ablation element. The ablation tip 30 may be connected via wires 32 and 34 to an energy source for enabling ablation. Alternatively, the ablation tip 30 may receive energy for ablating a tissue surface through other suitable means. The electrophysiology catheter 20 preferably comprises an ultrasound transducer 40 that may be connected via wires 42 to an energy source, and is capable of transmitting and receiving ultrasound pulses 44. An ultrasound transmitter device sends ultrasound pulses to the transducer 40, and a receiver device receives the pulses from the transducer 40. The data is subsequently transferred to a computer and used to define the location of the ultrasound catheter 20 and to provide imaging data of the operating area.

Alternatively, the catheter 20 may comprise an ultrasonic horn coupled to a waveguide which terminates at the distal tip 26 of the electrophysiology catheter 20. The horn may comprise a piezoelectric device for generating ultrasonic energy at frequencies up to 10 MHz, to be delivered to the waveguide to the distal tip 26 of the catheter. The high frequency ultrasonic waves may be transmitted to the ablation site and correspondingly detected by a transducer on the tip of the electrophysiology catheter. The transducer receives the ultrasonic pulse signals, which are then received by a computer. Any lesions formed by ablation can accordingly be imaged and displayed for determining if the line of ablation is satisfactory.

In operation, the ultrasonic imaging transducer 40 can be used to locate and visualize the lesion. Once the lesion has been located, the correct amount of power at a desired frequency can be delivered to the transducer 40 in the distal tip 24 of the ultrasonic imaging catheter 20 to improve imaging resolution of the lesion. The ultrasound image data can be suitably displayed to provide the physician with a projection of the ablated tissue during the procedure. The ultrasound imaging can be used to detect a point of unsatisfactory ablation along the line of ablation or lesion, and to redirect the ablation device to re-ablate the tissue at the location. The electrophysiology catheter 20 may further comprise one or more electrodes 52 for sensing tissue properties for mapping purposes.

Images from the ultrasound imaging from the distal end of the device can be displayed, for example on a navigational display or other display device. The physician or other health care worker can use the display image to decide upon, or more preferably to control the remote navigation system to orient the distal end 24 of the electrophysiology catheter 20. The physician may also be able to employ a preoperative image of the operating region, or more preferably a reconstruction of the operating region from preoperative imaging. Where there is only one branch opening 100 in the preoperative image or reconstruction in the vicinity of the operating region, the physician can verify that the opening in the ultrasound image corresponds to the opening shown in the preoperative image or reconstruction image on the display device. For example when navigating the medical device through a branching vessel, the device is advanced to a point near the branch and the ultrasound image is used to detect the opening 100 of the branch. Once the opening of the branch has been located, the remote navigation system 22 is operated to orient the distal end of the medical device toward the opening of the selected branch, so that the advancement of the device moves the device down the selected branch.

The ultrasonic images can be registered to the preoperative images or directly to the remote navigation system using magnetic localization of the distal tip of the medical device, which can provide position and orientation information of the distal tip so that the images from the distal tip can be used by the physician to control the remote navigation system. In another alternative, the ultrasonic images can be registered to the preoperative images or directly to the remote navigation system using external ultrasound imaging of the operating region. The position of the ultrasound transducer can be registered relative to the remote navigation system directly or registered to the remote navigation system via registration to the procedure suite.

Upon registration of the medical device with the remote navigation system as described above, the device can be navigated using only the ultrasound images from the medical device, without any external real time imaging of the operating region. The preoperative image, as well as real-time ultrasonic imaging from the ultrasonic imaging system can also be used to display an image of the device in the operating region to help orient the physician. This reduces the need for continual x-ray imaging, which while possible, is not necessary for successful navigation.

In another embodiment, the ultrasound imaging and ablation are performed by individual catheters as shown in FIG. 2. An ultrasound imaging catheter 20′ has a proximal end 22′ and a distal end 24′ and a lumen 26′ there between. The distal end 24′ of the ultrasound imaging catheter 20′ may further comprise a magnetically responsive element 28′, where magnetic navigation systems are employed. The ultrasound catheter 20′ preferably comprises an ultrasound transducer 40′ that may be connected via wires 42′ to an energy source, and is capable of transmitting and receiving ultrasound pulses 44. An ultrasound transmitter device sends ultrasound pulses to the transducer 40, and a receiver device receives the pulses from the transducer 40. The data is subsequently transferred to a computer and used to define the location of the ultrasound catheter 20′ to establish a reference frame, and to provide imaging data of the operating area.

An ablation catheter 50 is also provided that has a proximal and distal end, and an ablation tip 30 at the distal tip of the ablation catheter 50. The ablation tip 30 may be connected via wires 32 and 34 to an energy source for enabling ablation. Alternatively, the ablation tip 30 may receive energy for ablating a tissue surface through other suitable means. The distal end 24′ of the ablation catheter 50 may further comprise a magnetically responsive element 58, where magnetic navigation systems are employed. The ablation catheter 50 may further comprise one or more electrodes 52 for sensing tissue properties for mapping purposes. Accordingly, the ablation catheter 50 is preferably navigable by a physician to ablate tissue to form lesions.

The ultrasound reference catheter 20′ is equipped with at least one ultrasound transducer 40, and the ablation catheter 50 may also be equipped with at least one ultrasound transducer. The external ultrasound transmitter device sends ultrasound pulses to the transducers of the reference ultrasound catheter 20′ and ablation catheter 50. By measuring the time delay from the departure of a transmitted ultrasound pulse and the reception of this pulse at the other transducers, the distance between the individual transducers of the ultrasound and ablation catheters can be calculated. These data are subsequently transferred to the computer and used to define the location of the ultrasound catheter 20′ within a reference frame. Triangulation can be used to track the position of the ablation catheter 50 relative to the reference frame.

Upon registration of the ablation catheter 50 with the remote navigation system as described above, the ablation catheter 50 can be navigated using only the ultrasound images from the medical device, without any external real time imaging of the operating region. The preoperative image, as well as real-time ultrasonic imaging from the ultrasonic imaging system can also be used to display an image of the ablation catheter 50 in the operating region to help orient the physician. This reduces the need for continual x-ray imaging, which while possible, is not necessary for successful navigation.

In operation, the ultrasonic imaging transducer 40 can be used to locate and visualize the lesion. The ultrasound image data can be suitably displayed to provide the physician with a projection of the ablated tissue during the procedure. The ultrasound imaging can be used to detect a point of unsatisfactory ablation along the line of ablation or lesion. Once an unsatisfactory point in the lesion has been located, the ablation catheter can be navigated to the point and the correct amount of power for a given duration of time can be applied to provide a satisfactory ablation. The ultrasonic imaging catheter 20′ may be withdrawn a given distance from the ablation site during ablation, to prevent heat damage to the transducer 40 of the ultrasonic imaging catheter 20′.

In another embodiment, a method for automated control of ablation is provided, which could use both the burned tissue property and the neighboring blood velocity pattern as sensor signals. The tissue property would determine the mechanical effect of the ablation but the velocity pattern could be used to evaluate heart pumping performance. Both of these would require data banks to provide reasonably useful data for feedback. The two types of feedback would act in at least a “two loop control system.” The minor, interior loop would provide more direct and rapid feedback while the overall loop of the effectiveness of the heart pumping on the blood flow pattern would be a slower, less direct feedback. The reason for this is that multiple burns are often necessary to establish better heart pumping, and a single burn would not necessarily be the one which does that. Of course, the electrical wall signal sensors that are presently used to see timing patterns would be a possibly quicker signal of appropriate restoration of good timing. In the absence of any such flow pattern data, it is not known whether good signal timing is a complete and durable solution to arrhythmia.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A method of ablating tissue, the method comprising:
navigating an electrophysiology catheter to the site of the ablation;
applying an ablating electrode on the electrophysiology catheter to the tissue to be ablated;
applying energy to the electrode to ablate the tissue adjacent the electrode;
ultrasonically imaging the site of the ablation, and reablating the tissue if the ultrasound imaging does not show a satisfactory ablation.

2. The method according to claim 2 wherein the step of ultrasonically imaging the line of ablation is performed with a separate device from the electrophysiology catheter.

3. The method according to claim 2 wherein the step of ultrasonically imaging the lien of ablation is performed with the electrophysiology catheter.

4. A method of ablating tissue, the method comprising:
forming a line of ablation by successively navigating an electrophysiology catheter to the site of the ablation; applying an ablating electrode on the electrophysiology catheter to the tissue to be ablated; and applying energy to the electrode to ablate the tissue adjacent the electrode;
ultrasonically imaging the line of ablation to locate points on the line that do not show satisfactory ablation; and
navigating an electrophysiology catheter to at least one point on the line that does not show satisfactory ablation; applying an ablating electrode on the electrophysiology catheter to the tissue at the point; and applying energy to the electrode to ablate the tissue at the point.

5. The method according to claim 5 wherein the step of ultrasonically imaging the line of ablation is performed with a separate device from the electrophysiology catheter.

6. The method according to claim 5 wherein the step of ultrasonically imaging the line of ablation is performed with the electrophysiology catheter.

7. The method according to claim 5 wherein the step of navigating an electrophysiology catheter comprises automatically identifying at least one point on the line of ablation that does not show satisfactory ablation through image processing; and automatically navigating the electrophysiology catheter to the identified point with a remote navigation system.

8. The method according to claim 7 wherein the step of automatically navigating the electrophysiology catheter to the identified point is done with a magnetic navigation system.

9. The method according to claim 7 wherein the step of automatically navigating the electrophysiology catheter is done with a mechanical navigation system.

10. A method of ablating tissue, the method comprising:
applying an ablating electrode on the electrophysiology catheter to the tissue to be ablated and applying energy to the electrode to ablate the tissue adjacent the electrode until ultrasonic imaging of the site indicates satisfactory ablation.

11. A method of ablating tissue, the method comprising:
applying an ablating electrode on the electrophysiology catheter to the tissue to be ablated and
alternately applying energy to the electrode to ablate the tissue adjacent the electrode and ultrasonically imaging the site until the ultrasonic imaging indicates satisfactory ablation.

12. A method of treating arrhythmias, the method comprising ablating tissue to block errant electrical signals causing the arrhythmias, until ultrasonic measurement of flow velocities in the heart indicates that the arrhythmias has been attenuated.

13. A method of treating arrhythmias, the method comprising forming at least line of ablations to block errant signals causing the arrhythmias by ablating tissue until ultrasonic imaging indicates satisfactory ablation, and continuing the formation of lines until ultrasonic measurement of flow velocities in the heart indicates that the arrhythmias.

14. A method of treating arrhythmias, the method comprising forming at least one line of ablations to block errant signals causing the arrhythmias, the method comprising identifying a plurality of lines of ablation; sequentially forming each line by ablating tissue until ultrasonic imaging indicates satisfactory ablation, and continuing the formation of lines until ultrasonic measurement of flow velocities in the heart indicates that the arrhythmia has been attenuated.
